(19) United States
(12) Patent Application Publication
(10) Pub. No.: US 2003/0091518 A1
(43) Pub. Date: May 15, 2003

(54) Cosmetic and/or pharmaceutical preparations

(76) Inventors: Gilles Pauly, Nancy (FR); Philippe Moser, Essey-les-Nancy (FR); Philippe Moussou, Nancy (FR); Louis Danoux, Saulxures-les-Nancy (FR)

Correspondence Address: COGNIS CORPORATION, 2500 RENAISSANCE BLVD., SUITE 200, GULPH MILLS, PA, 19406

(21) Appl. No.: 10/168,636

(22) PCT Filed: Dec. 11, 2000
(86) PCT No.: PCT/EP00/12520

(30) Foreign Application Priority Data
Dec. 20, 1999 (FR) 99/16082
Jan. 31, 2000 (FR) 00/01218

(51) Int. Cl. A61K 35/78; A61K 7/42

(52) U.S. Cl. 424/59; 424/755; 514/514

(57) ABSTRACT

Cosmetic and/or pharmaceutical compositions which contain: (a) a Brassicaceae extract; and (b) one or more compounds selected from the group consisting of oil components, emulsifiers, antioxidants and UV/IR protection factors are described, along with methods for preparing and using the same.

### FIELD OF THE INVENTION

This invention relates generally to cosmetics and more particularly to new preparations containing an effective quantity of a certain plant extract or ingredients thereof together with oil components and/or emulsifiers and/or UV protection factors and/or antioxidants and to various uses of the extracts in cosmetics and pharmaceutical products.

### PRIOR ART

Cosmetic preparations are expected by consumers to meet increasingly more stringent requirements. In this connection, it goes without saying that a product intended, for example, to cleanse the skin should reliably perform that function. The user is also entitled to expect the composition of the product to have optimal dermatological compatibility so that even people with sensitive skin do not react with irritation. In addition, however, the preparations should also perform other functions which increasingly lie in the field of care and particularly protection. A particular concern among manufacturers of cosmetic and pharmaceutical preparations is the development of active ingredients which are readily accessible and can be produced at reasonable cost and which perform a whole range of functions, for example caring for the skin and hair and at the same time not only protecting them against the harmful effects of UV radiation, but also repairing existing damage. In actual fact, in fair-skinned people, the UV component of sunlight can cause more or less intensive skin irritation (“sunburn”) which can go as far as very serious burning. Even measured but repeated exposure is both dermatologically and cosmetically harmful because it is accompanied by accelerated ageing of the skin. This surface effect is generally accompanied by damage to the cell DNA and, in the worst case, can cause skin melanoma. These relationships have of course been known for time but this has only deterred a few genuine sunseekers from exposing themselves to the sun whenever climatic conditions and basic outdoor conditions permit and, if necessary, artificially acquiring a suntan, the use of self-tanning preparations being far safer than the permanent use of corresponding UV floodlamps.

Accordingly, consumers are constantly seeking new products which offer reliable protection against the dangers of UV radiation and allow increasingly longer exposure times. Increasing the sun protection factor by increasingly higher doses of the protection factors is not the answer because the stable incorporation of such substances in cosmetic preparations is always difficult and large amounts of active ingredients are synonymous with high costs. In some cases, the use of relatively high concentrations is not advisable because the filters can again cause irritation of the skin.

In this connection, reference is made to International patent application WO 99/20242 (Herba) which discloses dietetic or cosmetic preparations containing at least one heteropolysaccharide and at least one other component such as, for example, carotinoids and/or glucosinolates. Through the removal or fixing of heavy metals, these preparations have healing and protecting properties. These properties change relative to the removal of the heavy metals. Japanese patent application JP-A1 09/301833 (Sato) relates to the use of a mixture containing aloe, brown algae extract, broccoli extract and honey for the treatment of gray hair.

The problem addressed by the present invention was to provide cosmetic and/or pharmaceutical preparations of which the ingredients would not produce any skin irritation among users and would activate special repair and detoxification enzymes (for example glutathione-S-transferase), stimulate or regulate cell growth, influence the metabolic activity of fibroblasts or keratinocytes and could thus be used with advantage for the production of cosmetic and/or pharmaceutical preparations, especially skin and hair treatment preparations, without unwanted side effects, even in sensitive users. Another problem addressed by the invention was to make these cosmetic and/or pharmaceutical preparations available in particular as new sun protection preparations which—at one and the same time—would have a strong filter effect, would be photostable, could be incorporated easily and permanently in cosmetic formulations, even in high concentrations, would have optimal dermatological compatibility and, in addition, would exhibit both anti-inflammatory and skin-rejuvenating properties.

### DESCRIPTION OF THE INVENTION

The present invention relates to new cosmetic and/or pharmaceutical preparations which are characterized in that they contain

(a) an effective quantity of a Brassicaceae extract and

(b) oil components and/or emulsifiers and/or UV protection factors and/or antioxidants.

It has surprisingly been found that glucoraphanin and its isothiocyanate (sulforaphane), which occur in the extracts of plants of the genus Brassicaceae, especially in broccoli, universally meet the complex requirement profile mentioned above without causing unwelcome side effects. Another advantage of the products is their antimicrobial activity and their anti-inflammatory properties which may even be used to treat acne, especially Acne vulgaris. The invention also includes the observation that the preparations meet the complex requirement profile mentioned almost ideally in conjunction with commercially available UV protection factors or antioxidants. The preparations have a synergistically improved performance profile and improved photostability. The invention includes the further observation that the preparations counteract ageing of the skin and have a revitalizing and rejuvenating effect. In addition, the preparations increase the protection of skin and hair follicles against environmental toxins, oxidative stress and UV radiation, particularly UV-B radiation. Finally, they stimulate the collagen-forming fibroblasts and other molecules found in the dermis.

Brassicaceae Extracts

Crucifers of the genus Brassicaceae are characterized by a high content of mustard glycosides, so-called glucosinolates. Members of this plant family include, for example, rape, beets, all varieties of cabbage, radish, horseradish, capers, cresses, black and white mustard and wallflower. By virtue of their particularly high content of glucosinolates, extracts of broccoli or broccoli seeds and especially extracts of broccoli buds are particularly preferred for the purposes of the invention. The active substance identified in the extracts was the glucosinolate, glucoraphanin, which is readily converted into the isothiocyanate, sulforaphane, in the presence of myrosinase:

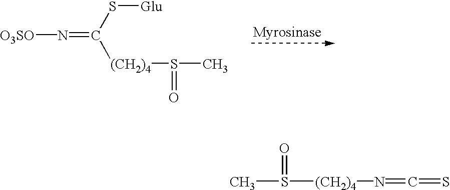

Accordingly, cosmetic preparations containing Brassicaceae or broccoli extracts on the one hand and glucoraphanin and/or sulforaphane on the other hand are united by the inventive concept that these two substances represent the active principles in the extracts. Thus, broccoli extract contains, for example, 30 to 35% and the extract of broccoli buds more than 70% of glucoraphanin out of the total content of glucosinolates in the buds. Reference is made by way of example in this connection to the work by Fahey et al. in Proc. Nat. Acad. Sci. USA 94, 10367 (1997) from which it is known that broccoli extract inhibits tumor genesis in rats. The effectiveness of glucosinolates in cancer prophylaxis is reported, for example, by Verhagen et al. in Carcinogenesis 16, 969 (1995). In particular, the use of sulforaphane, for example, in the detoxification of xenobiotic compounds for inhibiting cytochrome P450 enzymes and the like is known from the literature [cf. for example U.S. Pat. No. 5,411,986]. The use of broccoli as a lutein source is reported in Food Chem. 54, 101 (1995).

The extracts may be prepared in known manner, for example by the method described by Zhang et al. in Anal. Biochem. 205, 100 (1992). Particulars of other suitable extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux in a Soxhlet extractor, which are familiar to the expert and which may all be used in principle, can be found for example in Hagers Handbuch der pharmazeutischen Praxis (5th Edition, Vol. 2, pp. 1026-1030, Springer Verlag, Berlin-Heidelberg-New York 1991) The percolation method is advantageous for industrial application. Fresh plants or parts thereof are suitable as the starting material although dried or fresh plants, buds or seeds and/or plant parts which may be mechanically size-reduced before extraction are normally used. Any size reduction methods known to the expert, such as freeze grinding for example, may be used. Suitable solvents for the extraction process are organic solvents, water (preferably hot water with a temperature above 80° C. and, in particular, above 95° C.) or mixtures of organic solvents and water, more particularly low molecular weight alcohols with more or less large water contents. Extraction with methanol, ethanol, pentane, hexane, heptane, acetone, propylene glycols, polyethylene glycols and ethyl acetate, mixtures thereof and water-containing mixtures thereof is particularly preferred. The extraction process is generally carried out at 20 to 100° C., preferably at 30 to 90° C. and more particularly at 60 to 80° C. In one preferred embodiment, the extraction process is carried out in an inert gas atmosphere to avoid oxidation of the active principles of the extract. This is particularly important where extraction is carried out at temperatures above 40° C. The extraction times are selected by the expert in dependence upon the starting material, the extraction process, the extraction temperature and the ratio of solvent to raw material, etc. After the extraction process, the crude extracts obtained may optionaly be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of seeds, buds or plant parts are in the range from 3 to 30 and more particularly 5 to 25% by weight. The present invention includes the observation that the extraction conditions and the yields of the final extracts may be selected by the expert according to the desired application. After the extraction process, it is advisable to free the extracts from water by spray drying or freeze drying. The content of active principles in the extract may vary according to the raw material used. In addition, the content of active principles in the extract, based on the total quantity of extract, can be increased by enrichment or purification processes known to the expert as described, for example, by Kore et al. in J. Agric. Food Chem. 41, 89 (1993). The extracts typically have a content of 10 to 500, preferably 100 to 400 and more particulaly 200 to 300 μmol/g of the active principles. The active principles of the extracts exclusively or predominantly contain glucoraphane and/or sulforaphane. Mixtures of glucoraphane and sulforaphane are particularly preferred active principles for the purposes of the present invention. The percentage content of glucoraphanes in the extracts is generally higher by a factor of 4 to 10 than that of sulforaphanes. However, the glucosinolate can be hydrolyzed substantially completely to the isothiocyanate or mixtures of both active principles can be prepared by adding an effective quantity of thioglucosidase (myrosinase). The extracts may be used in quantities of 0.1 to 10% by weight, preferably in quantities of 0.5 to 5% by weight and more particularly in quantities of 1 to 2% by weight, based on the preparation.

In addition, the active principles may also be chemically, enzymatically or chemically/enzymatically prepared and used. Corresponding processes are described, for example, by Whitsell et al. in J. Org. Chem. 59, 597 (1994), by Schenk et al. in Chem. Eur. J. 3, 713 (1997), by Holland et al. in Tetrahedron: asymmetry 5, 1125 (1994) and by Iori et al. in Bioorg. Med. Chem. Lett. 9, 1047 (1999).

Oil Components

In a first embodiment of the present invention, the preparations may contain oil components in addition to the extracts. Suitable oil components are, for example, Guerbet alcohols based on fatty alcohols containing 6 to 18 and preferably 8 to 10 carbon atoms, esters of linear C₆₋₂₂ fatty acids with linear C₆₋₂₂ fatty alcohols, esters of branched C₆₋₁₃ carboxylic acids with linear C6-22 fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of hydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, Guerbet carbonates, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, ring opening products of epoxidized fatty acid esters with polyols, silicone oils and/or aliphatic or naphthenic hydrocarbons, for example squalane, squalene or dialkyl cyclohexanes.

In one particular embodiment of the invention, the preparations contain

(a) 0.1 to 10% by weight of extracts and

(b) 1 to 99.9% by weight of oil components and/or 0.1 to 15% by weight of emulsifiers, with the proviso that the the quantities shown optionally add up to 100% by weight with water and/or other auxiliaries and additives.

The percentage content of oil components in the preparations may be from 1 to 99.9% by weight and is preferably from 5 to 80% by weight and more particularly from 10 to 50% by weight, based on the preparations. A preparation falling within the scope of the invention may therefore contain, for example, from 0.1 to 10% by weight of extract and from 90 to 99.9% by weight of oil comoponents. If the quantities of extract and oil component together come to less than 100% by weight, the balance is made up by other ingredients—in the simplest case water.

Emulsifiers

The preparations according to the invention may additionally contain emulsifiers. Suitable emulsifiers are, for example, nonionic surfactants from at least one of the following groups:

products of the addition of 2 to 30 moles of ethylene oxide and/or 0 to 5 moles of propylene oxide onto linear C₈₋₂₂ fatty alcohols, C₁₂₋₂₂ fatty acids and alkyl phenols containing 8 to 15 carbon atoms in the alkyl group and alkylamines containing 8 to 22 carbon atoms in the alkyl group;

alkyl and/or alkenyl oligoglycosides containing 8 to 22 carbon atoms in the alkyl group and ethoxylated analogs thereof;

adducts of 1 to 15 moles of ethylene oxide with castor oil and/or hydrogenated castor oil;

adducts of 15 to 60 moles of ethylene oxide with castor oil and/or hydrogenated castor oil;

partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 moles of ethylene oxide;

partial esters of polyglycerol (average degree of self-condensation 2 to 8), polyethylene glycol (molecular weight 400 to 5000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 moles of ethylene oxide;

mixed esters of pentaerythritol, fatty acids, citric acid and fatty alcohol according to DE 11 65 574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

polyalkylene glycols and

glycerol carbonate.

The addition products of ethylene oxide and/or propylene oxide with fatty alcohols, fatty acids, alkylphenols or with castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as refatting agents for cosmetic formulations from DE 20 24 051 PS.

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 moles of ethylene oxide with the partial glycerides mentioned are also suitable.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxy-stearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 moles of ethylene oxide with the sorbitan esters mentioned are also suitable.

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof.

Examples of other suitable polyolesters are the mono-, di- and triesters of trimethylolpropane or pentaerythritol with lauric acid, coconut fatty acid, tallow fatty acid, palmitic acid, stearic acid, oleic acid, behenic acid and the like optionally reacted with 1 to 30 moles of ethylene oxide.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred. The percentage content of emulsifiers may be from 0.1 to 15% by weight and is preferably from 1 to 10% by weight and more particularly from 3 to 8% by weight. A preparation falling within the scope of the invention may therefore contain, for example, 0.1 to 10% by weight of extract and from 0.1 to 15% by weight of emulsifier. In the most simple case, the balance to 100% by weight is made up by water. Other additives—preferably emulsifiers—are described in more detail in the following.

UV/IR Protection Factors and Antioxidants

The preparations according to the invention may additionally contain UV or IR protection factors and/or antioxidants.

UV or IR protection factors in the context of the invention are, for example, organic substances (light filters) which are liquid or crystalline at room temperature and which are capable of absorbing ultraviolet or infrared radiation and of releasing the energy absorbed in the form of longer-wave radiation, for example heat. UV-B filters can be oil-soluble or water-soluble. The following are examples of oil-soluble substances:

3-benzylidene camphor or 3-benzylidene norcamphor and derivatives thereof, for example 3-(4-methylbenzylidene)-camphor as described in EP-B1 0693471;

4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;

esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);

esters of salicylic acid, preferably salicylic acid-2-ethylhexyl ester, salicylic acid-4-isopropylbenzyl ester, salicylic acid homomenthyl ester;

derivatives of benzophenone, preferably 2-hydroxy-4-methoxybenzo-phenone, 2-hydroxy-4-methoxy-4′-methylbenzophenone, 2,2′-dihydroxy-4-methoxybenzophenone;

esters of benzalmalonic acid, preferably 4-methoxybenzmalonic acid di-2-ethylhexyl ester;

triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (Uvasorb® HEB);

propane-1,3-diones such as, for example, 1-(4-tert.butylphenyl)-3-(4′-methoxyphenyl)-propane-1,3-dione;

ketotricyclo(5.2.1.0)decane derivatives as described in EP 0694521 B1.

Suitable water-soluble substances are

2-phenylbenzimidazole-5-sulfonic acid and alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and salts thereof;

sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and the enamine compounds described in DE 197 12 033 A1 (BASF). The UV-A and UV-B filters may of course also be used in the form of mixtures. Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or dimethicones. So-called micro- or nanopigments are preferably used in sun protection products. Micronized zinc oxide is preferably used. Other suitable UV filters can be found in P. Finkel's review in SÖFW-Journal 122, 543 (1996) and in Parfümerie und Kosmetik 3 (1999), pages 11 et seq.

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene, lutein) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, boldin, boldo extract, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

In one particular embodiment, the preparations contain

(a) 0.1 to 10% by weight of a Brassicaceae extract and

(b) 0.1 to 20% by weight of UV/IR protection factors and/or antioxidants,

with the proviso that the quantities shown add up to 100% by weight with water and/or other auxiliaries and additives.

The UV protection factors and/or antioxidants may be present in the preparations in quantities of 0.1 to 20% by weight, preferably 1 to 15% by weight and more particularly 3 to 10% by weight.

Commercial Applications

Both the Brassicaceae extracts in general and the broccoli extracts—above all the extracts of broccoli, broccoli seeds or broccoli buds—in particular and especially their active principles—glucoraphanin and sulforaphane—have a number of cosmetic and pharmaceutical effects. Accordingly, the present invention also relates to the use of Brassicaceae extracts, preferably broccoli extracts and in particular glucoraphanin and sulforaphane and above all mixtures thereof

as skin and hair care agents;

as anti-inflammatory agents;

as antimicrobial agents;

as antioxidants or radical scavengers;

as agents for stimulating or regulating skin cell formation;

as agents for stimulating skin detoxification enzymes, especially glutathione-S-transferase;

as anti-acne agents;

as skin rejuvenating agents;

as UV/IR protection factors;

as agents against fibroblast and/or keratinocyte damage by UV radiation, more particularly by UV-B radiation;

as agents against UV-induced apoptosis and against DNA damage, particularly by UV-B radiation.

If the Brassicaceae extracts are used as UV/IR protection factors, it is advisable to use other UV/IR protection factors and/or antioxidants which may be present in total quantities of 0.2 to 30% by weight, preferably 1 to 15% by weight and more particularly 5 to 10% by weight.

In the context of the invention, the terms “preparations” and “agents” are synonymous with the term “care preparations”.

Care preparations in the context of the invention are understood to be hair and skin care preparations. These care preparations have inter alia stimulating, healing and regenerating effects on the skin and hair. Preferred care preparations in the context of the invention are those which have a stimulating effect on the skin cells and their functions and a regenerating effect on the skin and hair and a protective effect against environmental influences on the skin and hair. Other preferred care preparations in the context of the invention are those which can either ameliorate or cure various diseases of the skin through their various effects on the appearance and function of the skin.

The preparations according to the invention have an excellent skin-care effect coupled with high dermatological compatibility. In addition, they show high stability, more particularly to oxidative decomposition of the products.

According to the invention, the Brassicaceae extracts act as anti-inflammatory care preparations which are capable of healing or preventing inflammation of the skin. Such inflammation can have various causes. In particular, the preparations according to the invention may be used to treat inflammation induced by UV radiation, contamination of the skin or bacterial and hormonal changes in the skin, for example acne.

According to the invention, the Brassicaceae extracts are active against ageing of the skin, above all against all forms of lining and wrinkling. The uses include the slowing down of skin ageing processes. The ageing signs can have various causes. In particular, they may be caused by UV-induced skin damage. In another embodiment of the invention, the Brassicaceae extracts are used against fibroblast and keratinocyte damage by UV radiation.

In another embodiment of the invention, the Brassicaceae extracts are used to treat UV-induced apoptosis and DNA damage and the signs of skin ageing thus induced.

In the context of the invention, apoptosis is understood to be the controlled cell death of certain unwanted or damaged cells. It is an active cel process (suicide on command). Apoptosis is initiated by oxidative stress (UV radiation, inflammation), by a deficiency of growth factors or by toxins (pollutants, genotoxins, etc.). In the skin ageing process, for example, apoptosis of the skin cells can be induced by a deficiency of growth factors in the skin. In the apoptosis-affected cells, the nuclear DNA is degraded by the specific enzyme endonuclease and the DNA fragments are channeled into the cytoplasm. Apoptosis can also be induced by UV radiation, especially UV-B radiation. In principle, growth factors are understood to be genetic or extrinsic growth factors which stimulate the growth of skin and hair cells. They include, for example, hormones and chemical mediators or signal molecules. Examples are polypeptide growth factors and glycoprotein growth factors. Mention is made here of the epidermal growth factor (EGF), which consists of 53 amino acids and hence represents a polypeptide growth factor, or fibrillin which is a glycoprotein. Other growth factors are, for example, urogastrone, laminin, follistatin and heregelin.

Besides the above-mentioned effects of the Brassicaceae extracts, positive effects have also been observed in the control of melanogenesis. These effects allow Brassicaceae extracts to be used as skin whiteners or to prevent hair from turning gray. Effects have also been observed in the activation of lipolysis. These effects enable the Brassicaceae extracts according to the invention to be used as anti-cellulite agents and as slimming preparations. Their use as agents for increasing the elasticity and firmness of the skin derives from the effects observed in the investigation of antiprotease activity. The Brassicaceae extracts according to the invention show anti-collagenase and anti-elastase activity and, accordingly, counteract destruction of the skin proteins critically involved in the development of elasticity and firmness of the skin by the enzymes collagenase and elastase.

Cosmetic and/or Pharmaceutical Preparations

The extracts or active principles may be may be used for the production of cosmetic and/or pharmaceutical preparations such as, for example, hair shampoos, hair lotions, foam baths, shower baths, creams, gels, lotions, alcoholic and aqueous/alcoholic solutions, emulsions, wax/fat compounds, stick preparations, powders or ointments. Besides the oil components, emulsifiers and UV/IR protection factors and/or antioxidants mentioned above, these preparations may also contain mild surfactants, superfatting agents, pearlizing waxes, consistency factors, thickeners, polymers, silicone compounds, fats, waxes, lecithins, phospholipids, stabilizers, biogenic agents, deodorants, antiperspirants, antidandruff agents, film formers, swelling agents, hydrotropes, preservatives, insect repellents, self-tanning agents, tyrosine inhibitors (depigmenting agents), solubilizers, perfume oils, dyes and the like as further auxiliaries and additives.

Typical examples of suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines and/or protein fatty acid condensates, preferably based on wheat proteins.

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used.

Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol mono-esters and diesters of fatty acids, polyacrylates (for example Carbopols® [Goodrich] or Synthalens® [Sigma]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryidimonium Hydroxypropyl Hydrolyzed Collagen (Lamequat® L, Grünau), quaternized wheat polypeptides, polyethyleneimine, cationic silicone polymers such as, for example, Amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2 252 840 A and crosslinked water-soluble polymers thereof, cationic chitin derivatives such as, for example, quaternized chitosan, optionally in microcrystalline distribution, condensation products of dihaloalkylene, for example dibromobutane, with bis-dialkylamines, for example bis-dimethylamino-1,3-propane, cationic guar gum such as, for example, Jaguar® CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Typical examples of fats are glycerides while suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes, microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycero-phospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Metal salts of fatty acids such as, for example, magnesium, aluminium and/or zinc stearate or ricinoleate may be used as stabilizers.

In the context of the invention, biogenic agents are, for example, tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, other plant extracts and vitamin complexes.

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Basically, suitable germ inhibitors are any substances which act against gram-positive bacteria such as, for example, 4-hydroxybenzoic acidand salts and esters thereof, N-(4-chlorophenyl)-N′-(3,4-dichloro-phenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chloro-phenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monolaurate (GML), diglycerol monocaprate (DMC), salicylic acid-N-alkylamides such as, for example, salicylic acid-n-octyl amide or salicylic acid-n-decyl amide.

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT, Henkel KGaA, Düsseldorf, FRG). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of ladanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexyl-cinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romilat, irotyl and floramat.

Antiperspirants reduce perspiration and thus counteract underarm wetness and body odor by influencing the activity of the eccrine sweat glands. Aqueous or water-free antiperspirant formulations typically contain the following ingredients:

astringent active principles,

oil components,

nonionic emulsifiers,

co-emulsifiers,

consistency factors,

auxiliaries in the form of, for example, thickeners or complexing agents and/or

nonaqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,

inflammation-inhibiting, skin-protecting or pleasant-smelling essential oils,

synthetic skin-protecting agents and/or

oil-soluble perfume oils.

Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Suitable antidandruff agents are Octopirox® (1-hydroxy-4-methyl-6-(2,4,4-trimethylpentyl)-2-(1H)-pyridinone monoethanolamine salt), Baypival, Piroctone Olamine, Ketoconazole® (4-acetyl-1-{4-[2-(2,4-dichlorophenyl) r-2-(1H-imidazol-1-ylmethyl)-1,3-dioxylan-c-4-ylmethoxy-phenyl}-piperazine, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”). Suitable insect repellents are N,N-diethyl-m-toluamide, pentane-1,2-diol or Ethyl Butylacetyl-aminopropionate. A suitable self-tanning agent is dihydroxyacetone. Suitable tyrosine inhibitors which prevent the formation of melanin and are used in depigmenting agents are, for example, arbutin, ferulic acid kojic acid, coumaric acid and ascorbic acid (vitamin C).

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamon, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable dyes are any of the substances suitable and approved for cosmetic purposes as listed, for example, in the publication “Kosmetische Färbemittel” of the Farbstoffkommission der Deutschen Forschungs-gemeinschaft, Verlag Chemie, Weinheim, 1984, pages 81 to 106. These dyes are normally used in concentrations of 0.001 to 0.1% by weight, based on the mixture as a whole.

The total percentage content of auxiliaries and additives may be from 1 to 50% by weight and is preferably from 5 to 40% by weight, based on the particular preparation. The preparations may be produced by standard hot or cold processes and are preferably produced by the phase inversion temperature method.

### Examples

### Preparation Example H1

0.4 kg deep-frozen 3.8-day-old broccoli buds were ground and dispersed in 800 ml water. The suspension was stirred for 4 h at 20° C., centrifuged and finally filtered to remove insoluble constituents. The resulting aqueous extract was heated for 15 mins. to 102° C., concentrated under reduced pressure and finally freeze-dried.

### Preparation Example H2

0.15 kg of deep-frozen 3.8-day-old broccoli buds were ground and dispersed in 225 ml of water. 1200 ml of methanol were then added and the mixture was heated under reflux for 1 hour and extracted in the process. The suspension was then cooled, filtered and the solvent distilled off under reduced pressure. The resulting concentrate was freeze-dried and had a content of 220 μmol/g glucosinolates and 13 μmol/g isothiocyanates.

### Preparation Example H3

5.2 g of the freeze-dried extract from Example H2 were suspended in 100 g of water and the resulting suspension was adjusted to pH 6 by addition of 5N sodium hydroxide solution. 25 U thioglucosidase (1 U=quantity of enzyme needed to prepare 1 μmol of glucose from sinigrin at 25° C./pH 6) and 9.9 mg of sodium ascorbate were added and the mixture was stirred for 8 h at 37° C. The pH was then readjusted to pH 6 by addition of more sodium hydroxide and the suspension was filtered and freeze-dried. The isothiocyanate content of the extract was 230 μmol/g.

### Preparation Example H4

0.2 kg broccoli seeds were suspended in 0.4 kg of water. 1600 ml of methanol were then added and the mixture was heated under reflux for 1 hour and extracted in the process. The suspension was then cooled and filtered and the solvent was distilled off under reduced pressure. The resulting concentrate was freeze-dried and had a content of 195 μmol/g glucosinolates and 59 μmol/g isothiocyanates.

The glucosinolates and the isothiocyanates were all quantified by Zhang's method (Y. Zhang et al., Anal. Biochem. 205, 100-107 (1992).

### Example 5

### Skin Regenerating and Revitalizing Activity

The object of this test is to demonstrate the regenerating and revitalizing activity of extracts of broccoli sprouts on human fibroblast cultures in vitro.

Method 1: Effects on Cell Growth

Human fibroblasts were inoculated with 10% by weight fetal calf serum in a defined nutrient medium (DMEM=Dulbecco Minimum Essential Medium, a product of Life Technologie S.a.r.l.) and incubated for 24 h at 37° C. in a 5% CO₂ atmosphere. The nutrient medium containing fetal calf serum was then replaced by a nutrient medium of DMEM without fetal calf serum. Broccoli bud extracts from Examples H1 to H3 were then added to this nutrient medium in various concentrations. For comparison, a test series of human fibroblasts with no active substance was incubated as control. After the fibroblasts had been incubated for three days in the nutrient medium, growth and metabolic activity were evaluated by counting the cells with a particle counter and determining the intracellular content of proteins by Bradford's method (Anal. Biochem. 1976, 72, 248-254) and of ATP by Vasseur's method (Journal Francais Hydrologie, 1981, 9, 149-156). With concentrations of 0.001 to 0.03% by weight of extract of Examples H1 to H3, an increase in the percentage ATP content of up to 16% was obtained by comparison with the control.

The study shows that the broccoli extracts of Examples H1 to H3 stimulate the growth and metabolism of the human fibroblasts in vitro.

Method 2: Improvement of Viability

The test was carried out on human fibroblasts. It enables a certain number of parameters to be quantitatively determined on the resting cells. The cultivation of the cells corresponds to the cultivation of method 1 except for the incubation time. The incubation time for this test was 72 h. Viability was evaluated by colorimetric determination of the percentage protein content by Bradford's method (Anal. Biochem. 1976, 72, 248-254), by determination of the percentage glutathione content (GSH) with a fluorescent probe, orthophthaldehyde, by Hissin and Hilf's method (Anal. Biochem. 1976, 74, 214-216) and via the mitochondrial activity MTT by the method described in J. Immunol. Methods 89, 271 (1987). The glutathione is produced by cells in order to be able to react directly against oxidative stress and environmental influences, such as high heavy metal levels. Accordingly, an increased percentage content of reduced glutathione after treatment of the cells with the extracts of Examples H1 to H3 is a measure of the increased viability of the cells under the effect of external stress and other challenges. The test was carried out three times and then repeated twice so that there were six results per plant extract and hence per batch which were all averaged. The results were expressed in percent by comparison with the control.

The results are set out in Table 1. The activity data represent percentages based on the blank value.

TABLE 1
In vitro growth and viability test on fibroblasts

| Concentration [% w/v] | Proteins | ATP | Proteins | MTT | GSH |
|---|---|---|---|---|---|
| None | 100 | 100 | 100 | 100 | 100 |
| Extract, Preparation Example 1 | | | | | |
| −0.001 | 103 | 101 | 103 | | |
| −0.003 | 113 | 89 | 113 | | |
| −0.005 | | | | 100 | 98 |
| −0.01 | 113 | 90 | 113 | 81 | 111 |
| −0.02 | | | | 72 | 126 |
| Extract, Preparation Example H2 | | | | | |
| −0.001 | 108 | 113 | 108 | | |
| −0.003 | 112 | 102 | 112 | | |
| −0.01 | 120 | 116 | 120 | 113 | 107 |
| −0.02 | | | | 131 | 125 |
| −0.03 | 131 | 94 | 131 | 147 | 149 |
| Extract, Preparation Example 3 | | | | | |
| −0.005 | | | | 106 | 91 |
| −0.01 | | | | 113 | 114 |
| −0.02 | | | | 138 | 193 |

The results set out in the Table offer clear proof of a growth-promoting effect of the extracts on the treated fibroblasts through the increased content of proteins and adenosine triphosphate (ATP), more particularly after treatment with the extracts of Preparation Example 2.

The improvement in viability was demonstrated by an increase in the GSH and MTT contents after treatment of the fibroblasts with the extracts.

### Example 6

In order to investigate the anti-inflammatory effect, PMN preparations (polymorphonuclear neutrophilic granulocytes) were incubated with the extracts for 24 h (37° C./5% CO₂) [cf. J. Invest. Dermatol. 95, 94S 1999); Immunopharmacology 23, 191 (1992)]. A yeast extract was then added to the cell suspensions and the preparations were incubated for another 30 mins. under the same conditions. The quantity of leucocytes was then determined using an automatic cell counter, the RSA (released superoxide anions) was quantified with luminol. The results are set out in Table 2. The activity data again represent percentages based on the blank value.

TABLE 2
Anti-inflammatory effect on leucocyte cells

| Concentration [% w/v] | Leucocytes | RSA |
|---|---|---|
| None | 100 | 100 |
| Extract, Preparation Example 1 | | |
| −0.001 | 103 | 86 |
| −0.01 | 99 | |
| −0.1 | 101 | 31 |
| Extract, Preparation Example 2 | | |
| −0.001 | 101 | 63 |
| −0.01 | 104 | 15 |
| −0.1 | 103 | 1 |
| Extract, Preparation Example 3 | | |
| −0.001 | 101 | 61 |
| −0.01 | 104 | 33 |
| −0.1 | 103 | 2 |

The results set out in the Table reflect a clear anti-inflammatory effect of the extracts investigated because, for the same number of keratinocytes, the number of free superoxide anions (RSA) is greatly reduced with increasing concentration of the extract. These results show that the extracts are non-toxic to the cells and that the content of inflammation-promoting RSA is distinctly reduced.

### Example 7

In order to determine effectiveness against UV-B radiation, cultures of human keratinocytes were incubated for 72 h (37° C./5% CO₂) in nutrient media. The nutrient media were then exchanged for salt solutions which contained corresponding quantities of the extracts to be tested. The preparations were exposed to UV-B radiation (50 mJ/cm², DUKE GL40E lamp) and incubated for another 24 h under the conditions mentioned. The keratinocyte count was determined after trypsination; released lactate dehydrogenase (LDH) was measured spectroscopically as a measure of the cell damage [cf. Photochem. Photobiol. 41(1), 51 (1985); Dermatol. Res. 282, 325 (1990)]. The results are set out in Table 3. The activity data again represent percentages based on the blank value.

TABLE 3
Effectiveness against UV-B radiation

| Concentration [% w/v] | Keratinocytes | LDH |
|---|---|---|
| None (no UV-B radiation) | 100 | 0 |
| None (with UV-B radiation) | 25 | 100 |
| Extract, Preparation Example 2 | | |
| −0.01 | 63 | 31 |
| −0.02 | 69 | 27 |
| Extract, Preparation Example 3 | | |
| −0.0025 | 43 | 59 |
| −0.005 | 46 | 39 |
| −0.01 | 54 | 29 |

### Example 8

### Cell Protecting Effect Against UV-A on Human Fibroblasts Cultivated In vitro

Background: UV-A rays (from 320 to 400 nm) penetrate into the dermis where they lead to oxidative stress which is demonstrated by lipoperoxidation of the cytoplasm membranes.

The lipoperoxides are degraded to malonaldialdehyde which will crosslink many biological molecules, such as proteins and nuclein bases (enzyme inhibition or mutagenesis).

Method: To carry out these tests, a defined culture medium containing the fibroblasts is inoculated with fetal calf serum and added to the plant extract (in the defined medium containing 2% serum) 72 hours after inoculation.

After incubation for 48 hours at 37° C./5% CO₂, the culture medium was replaced by a sodium chloride solution and the fibroblasts were exposed to a dose of UV-A (365 nm, 15 J/cm²; tubes: MAZDA FLUOR TFWN40).

After the exposure to UV-A, the MDA level (malonaldialdehyde level) in the supernatant sodium chloride solution was quantitatively determined by reaction with thiobarbituric acid. Besides the MDA level, the protein content was also determined.

TABLE 4
Quantification of malonaldialdehyde in fibroblasts (results in %, based on
the control, average value of 2 tests each repeated three times)

| Concentration (% w/v) | MDA level | Protein content |
|---|---|---|
| Control without UV | 0 | 0 |
| UV-A (365 nm) | 100 | 97 |
| UV-A + extract of Example H4, 0.03% | 45 | 118 |
| UV-A + extract of Example H4, 0.1% | 36 | 132 |

The results set out in Table 4 show that the extracts according to the invention significantly reduce the level of MDA in human fibroblasts which is induced by UV-A radiation. These results reflect a high capacity on the part of broccoli seed extracts to reduce the harmful effects of oxidative stress on the skin or on the hair follicles.

### Example 9

### Effectiveness Against UV-B-induced Apoptosis and DNA Damage in Human Keratinocytes Cultivated In vitro

Background: Apoptosis (cell death) can be induced by UV-B radiation. These cells show an increased level of destroyed DNA which is degraded by endonuclease. DNA fragments are left in the cytoplasm.

Method: In order to demonstrate induced apoptosis and induced DNA damage by UV-B radiation, human keratinocytes were investigated. To carry out the tests, a defined medium (DMEM=Dulbecco Minimum Essential Medium, a product of Life Technologie S.a.r.l.) which contains 10% fetal calf serum was inoculated with the keratinocytes, incubated at 37° C./5% CO₂ and added to the extract of Examples H2 and H3 (diluted with sodium chloride solution) 72 hours after inoculation.

The keratinocytes were then exposed to a dose of UV-B (50 mJ/cm²—tubes: DUKE GL40E) and incubated for another 24 hours at 37° C./5% CO₂.

The humber of adhering keratinocytes was determined (after trypsin treatment) with a particle counter. The content of DNA fragments in the cytoplasm was then determined by Parat's method (Parat et al., J. Photochem. Photobiol. B. Biol. 37, 101, 1997).

TABLE 5
Content of free DNA fragments in keratinocytes after
apoptosis induced by UV-B radiation

| | | | Content |
|---|---|---|---|
| | | | of DNA |
| | Concentration | Keratinocyte | frag- |
| | (% w/v) | count | ments |
| Control without UV-B | | 100 | 0 |
| Control with UV-B | | 97 | 100 |
| Extract, Preparation Example H2 | 0.01 | 126 | 55 |
| Extract, Preparation Example H3 | 0.005 | 123 | 71 |

The results prove that the extracts to be tested reduce the content of free DNA fragments in the cytoplasm of keratinocytes where apoptosis has been induced by UV-B radiation. It was thus possible to demonstrate that the destruction of DNA was prevented to a very considerable extent by the extracts after the UV-B radiation. The extracts are suitable as agents against UV-B-induced apoptosis and as agents against UV-B-induced DNA damage in human skin cells and hair follicles.

### Example 10

In order to determine antimicrobial activity, 6 mm large pieces of filter paper impregnated with 20 μl of various test solutions (0.1%) were applied to the surface of an agar preparation to which Propionibacterium acnes had been freshly added (1.5 10⁶ bacteria/ml). Effectiveness was investigated by determining the average diameter of the zones in which no bacterial growth could be detected. The results are set out in Table 6.

TABLE 6
Effectiveness against acne bacteria
(expressed as inhibition zone diameter in mm)

| Concentration [% w/v] | Extract, Example 2 | Extract, Example 3 |
|---|---|---|
| 0.1 | 8 | 9 |

The 8 mm and 9 mm inhibition zones show distinct inhibition of the growth of Propionibacterium acnes in the area around the filter papers impregnated with the extracts. This is proof of the growth-inhibiting effect of the extracts on a potential acne producer.

### Example 11

### Formulation Examples

A number of Formulation Examples are set out in Table 7.

TABLE 7
Cosmetic preparations (water, preservative to 100% by weight)

| Composition (INCI) | 1 | 2 | 3 | 4 | 5 | 6 | 7 | 8 | 9 | 10 |
|---|---|---|---|---|---|---|---|---|---|---|
| Texapon ® NSO | — | — | — | — | — | — | 38.0 | 38.0 | 25.0 | — |
| Sodium Laureth Sulfate | | | | | | | | | | |
| Texapon ® SB 3 | — | — | — | — | — | — | — | — | 10.0 | — |
| Disodium Laureth Sulfosuccinate | | | | | | | | | | |
| Plantacare ® 818 | — | — | — | — | — | — | 7.0 | 7.0 | 6.0 | — |
| Coco Glucosides | | | | | | | | | | |
| Plantacare ® PS 10 | — | — | — | — | — | — | — | — | — | 16.0 |
| Sodium Laureth Sulfate (and) Coco Glucosides | | | | | | | | | | |
| Dehyton ® PK 45 | — | — | — | — | — | — | — | — | 10.0 | — |
| Cocamidopropyl Betaine | | | | | | | | | | |
| Dehyquart ® A | 2.0 | 2.0 | 2.0 | 2.0 | 4.0 | 3.0 | — | — | — | — |
| Cetrimonium Chloride | | | | | | | | | | |
| Dehyquart L ® 80 | 1.2 | 1.2 | 1.2 | 1.2 | 0.6 | 0.6 | — | — | — | — |
| Dococoylmethylethoxymonium Methosulfate (and) | | | | | | | | | | |
| Propyleneglycol | | | | | | | | | | |
| Eumulgin ® B2 | 0.8 | 0.8 | — | 0.8 | — | 1.0 | — | — | — | — |
| Ceteareth-20 | | | | | | | | | | |
| Eumulgin ® VL 75 | — | — | 0.8 | — | 0.8 | — | — | — | — | — |
| Lauryl Glucoside (and) Polyglyceryl-2 | | | | | | | | | | |
| Polyhydroxystearate (and) Glycerin | | | | | | | | | | |
| Lanette ® O | 2.5 | 2.5 | 2.5 | 2.5 | 3.0 | 2.5 | — | — | — | — |
| Cetearyl Alcohol | | | | | | | | | | |
| Cutina ® GMS | 0.5 | 0.5 | 0.5 | 0.5 | 0.5 | 1.0 | — | — | — | — |
| Glyceryl Stearate | | | | | | | | | | |
| Cetiol ® HE | 1.0 | — | — | — | — | — | — | — | 1.0 | |
| PEG-7 Glyceryl Cocoate | | | | | | | | | | |
| Cetiol ® PGL | — | 1.0 | — | — | 1.0 | — | — | — | — | — |
| Hexyldecanol and Hexyldecyl laurate | | | | | | | | | | |
| Cetiol ® V | — | — | — | 1.0 | — | — | — | — | — | — |
| Decyl Oleate | | | | | | | | | | |
| Eutanol ® G | — | — | 1.0 | — | — | 1.0 | — | — | — | — |
| Octyldodecanol | | | | | | | | | | |
| Nutrilan ® Keratin W | — | — | — | 2.0 | — | — | — | — | — | — |
| Hydrolyzed Keratin | | | | | | | | | | |
| Lamesoft ® LMG | — | — | — | — | — | — | 3.0 | 2.0 | 4.0 | — |
| Glyceryl Laurate (and) Potassium Cocoyl | | | | | | | | | | |
| Hydrolyzed Collagen | | | | | | | | | | |
| Euperlan ® PK 3000 AM | — | — | — | — | — | — | — | 3.0 | 5.0 | 5.0 |
| Glycol Distearate (and) Laureth-4 (and) | | | | | | | | | | |
| Cocamidopropyl Betaine | | | | | | | | | | |
| Generol ® 122 N | — | — | — | — | 1.0 | 1.0 | — | — | — | — |
| Soya Sterol | | | | | | | | | | |
| Broccoli bud extract H1 | 1.0 | 1.0 | 1.0 | 1.0 | 1.0 | 1.0 | 1.0 | 1.0 | 1.0 | 1.0 |
| Hydagen ® HCMF | 1.0 | 1.0 | 1.0 | 1.0 | 1.0 | 1.0 | 1.0 | 1.0 | 1.0 | 1.0 |
| Chitosan | | | | | | | | | | |
| Copherol ® 12250 | — | — | 0.1 | 0.1 | — | — | — | — | — | — |
| Tocopherol Acetate | | | | | | | | | | |
| Arlypon ® F | — | — | — | — | — | — | 3.0 | 3.0 | 1.0 | — |
| Laureth-2 | | | | | | | | | | |
| Sodium Chloride | — | — | — | — | — | — | — | 1.5 | — | 1.5 |
| Composition (INCI) | 11 | 12 | 13 | 14 | 15 | 16 | 17 | 18 | 19 | 20 |
| Texapon ® NSO | 20.0 | 20.0 | 12.4 | — | 25.0 | 11.0 | — | — | — | — |
| Sodium Laureth Sulfate | | | | | | | | | | |
| Texapon ® K 14 S | — | — | — | — | — | — | — | — | 11.0 | 23.0 |
| Sodium Myreth Sulfate | | | | | | | | | | |
| Texapon ® SB 3 | — | — | — | — | — | 7.0 | — | — | — | — |
| Disodium Laureth Sulfosuccinate | | | | | | | | | | |
| Plantacare ® 818 | 5.0 | 5.0 | 4.0 | — | — | — | — | — | 6.0 | 4.0 |
| Coco Glucosides | | | | | | | | | | |
| Plantacare ® 2000 | — | — | — | — | 5.0 | 4.0 | — | — | — | — |
| Decyl Glucoside | | | | | | | | | | |
| Plantacare ® PS 10 | — | — | — | 40.0 | — | — | 16.0 | 17.0 | — | — |
| Sodium Laureth Sulfate (and) Coco Glucosides | | | | | | | | | | |
| Dehyton ® PK 45 | 20.0 | 20.0 | — | — | 8.0 | — | — | — | — | 7.0 |
| Cocamidopropyl Betaine | | | | | | | | | | |
| Eumulgin ® B1 | — | — | — | — | 1.0 | — | — | — | — | — |
| Ceteareth-12 | | | | | | | | | | |
| Eumulgin ® B2 | — | — | — | 1.0 | — | — | — | — | — | — |
| Ceteareth-20 | | | | | | | | | | |
| Lameform ® TGI | — | — | — | 4.0 | — | — | — | — | — | — |
| Polyglyceryl-3 Isostearate | | | | | | | | | | |
| Dehymuls ® PGPH | — | — | 1.0 | — | — | — | — | — | — | — |
| Polyglyceryl-2 Dipolyhydroxystearate | | | | | | | | | | |
| Monomuls ® 90-L 12 | — | — | — | — | — | — | — | — | 1.0 | 1.0 |
| Glyceryl Laurate | | | | | | | | | | |
| Cetiol ® HE | — | 0.2 | — | — | — | — | — | — | — | — |
| PEG-7 Glyceryl Cocoate | | | | | | | | | | |
| Eutanol ® G | — | — | — | 3.0 | — | — | — | — | — | — |
| Octyldodecanol | | | | | | | | | | |
| Nutrilan ® Keratin W | — | — | — | — | — | — | — | — | 2.0 | 2.0 |
| Hydrolyzed Keratin | | | | | | | | | | |
| Nutrilan ® I | 1.0 | — | — | — | — | 2.0 | — | 2.0 | — | — |
| Hydrolyzed Collagen | | | | | | | | | | |
| Lamesoft ® LMG | — | — | — | — | — | — | — | — | 1.0 | — |
| Glyceryl Laurate (and) Potassium Cocoyl | | | | | | | | | | |
| Hydrolyzed Collagen | | | | | | | | | | |
| Lamesoft ® 156 | — | — | — | — | — | — | — | — | — | 5.0 |
| Hydrogenated Tallow Glyceride (and) | | | | | | | | | | |
| Potassium Cocoyl Hydrolyzed Collagen | | | | | | | | | | |
| Gluadin ® WK | 1.0 | 1.5 | 4.0 | 1.0 | 3.0 | 1.0 | 2.0 | 2.0 | 2.0 | — |
| Sodium Cocoyl Hydrolyzed Wheat Protein | | | | | | | | | | |
| Euperlan ® PK 3000 AM | 5.0 | 3.0 | 4.0 | — | — | — | — | 3.0 | 3.0 | — |
| Glycol Distearate (and) Laureth-4 (and) | | | | | | | | | | |
| Cocamidopropyl Betaine | | | | | | | | | | |
| Panthenol | — | — | 1.0 | — | — | — | — | — | — | — |
| Arlypon ® F | 2.6 | 1.6 | — | 1.0 | 1.5 | — | — | — | — | — |
| Laureth-2 | | | | | | | | | | |
| Broccoli bud extract H3 | 1.0 | 1.0 | 1.0 | 1.0 | 1.0 | 1.0 | 1.0 | 1.0 | 1.0 | 1.0 |
| Hydagen ® CMF | 1.0 | 1.0 | 1.0 | 1.0 | 1.0 | 1.0 | 1.0 | 1.0 | 1.0 | 1.0 |
| Chitosan | | | | | | | | | | |
| Sodium Chloride | — | — | — | — | — | 1.6 | 2.0 | 2.2 | — | 3.0 |
| Glycerin (86% by weight) | — | 5.0 | — | — | — | — | — | 1.0 | 3.0 | — |
| Composition (INCI) | 21 | 22 | 23 | 24 | 25 | 26 | 27 | 28 | 29 | 30 |
| Texapon ® NSO | — | 30.0 | 30.0 | — | 25.0 | — | — | — | — | — |
| Sodium Laureth Sulfate | | | | | | | | | | |
| Plantacare ® 818 | — | 10.0 | — | — | 20.0 | — | — | — | — | — |
| Coco Glucosides | | | | | | | | | | |
| Plantacare ® PS 10 | 22.0 | — | 5.0 | 22.0 | — | — | — | — | — | — |
| Sodium Laureth Sulfate (and) Coco Glucosides | | | | | | | | | | |
| Dehyton ® PK 45 | 15.0 | 10.0 | 15.0 | 15.0 | 20.0 | — | — | — | — | — |
| Cocamidopropyl Betaine | | | | | | | | | | |
| Emulgade ® SE | — | — | — | — | — | 5.0 | 5.0 | 4.0 | — | — |
| Glyceryl Stearate (and) Ceteareth 12/20 | | | | | | | | | | |
| (and) Cetearyl Alcohol (and) Cetyl Palmitate | | | | | | | | | | |
| Eumulgin ® B1 | — | — | — | — | — | — | — | 1.0 | — | — |
| Ceteareth-12 | | | | | | | | | | |
| Lameform ® TGI | — | — | — | — | — | — | — | — | 4.0 | — |
| Polyglyceryl-3 Isostearate | | | | | | | | | | |
| Dehymuls ® PGPH | — | — | — | — | — | — | — | — | — | 4.0 |
| Polyglyceryl-2 Dipolyhydroxystearate | | | | | | | | | | |
| Monomuls ® 90-O 18 | — | — | — | — | — | — | — | — | 2.0 | — |
| Glyceryl Oleate | | | | | | | | | | |
| Cetiol ® HE | 2.0 | — | — | 2.0 | 5.0 | — | — | — | — | 2.0 |
| PEG-7 Glyceryl Cocoate | | | | | | | | | | |
| Cetiol ® OE | — | — | — | — | — | — | — | — | 5.0 | 6.0 |
| Dicaprylyl Ether | | | | | | | | | | |
| Cetiol ® PGL | — | — | — | — | — | — | — | 3.0 | 10.0 | 9.0 |
| Hexyldecanol (and) Hexyldecyl Laurate | | | | | | | | | | |
| Cetiol ® SN | — | — | — | — | — | 3.0 | 3.0 | — | — | — |
| Cetearyl Isononanoate | | | | | | | | | | |
| Cetiol ® V | — | — | — | — | — | 3.0 | 3.0 | — | — | — |
| Decyl Oleate | | | | | | | | | | |
| Myritol ® 318 | — | — | — | — | — | — | — | 3.0 | 5.0 | 5.0 |
| Coco Caprylate Caprate | | | | | | | | | | |
| Bees Wax | — | — | — | — | — | — | — | — | 7.0 | 5.0 |
| Nutrilan ® Elastin E20 | — | — | — | — | — | 2.0 | — | — | — | — |
| Hydrolyzed Elastin | | | | | | | | | | |
| Nutrilan ® I-50 | — | — | — | — | 2.0 | — | 2.0 | — | — | — |
| Hydrolzed Collagen | | | | | | | | | | |
| Gluadin ® AGP | 0.5 | 0.5 | 0.5 | — | — | — | — | 0.5 | — | — |
| Hydrolyzed Wheat Gluten | | | | | | | | | | |
| Gluadin ® WK | 2.0 | 2.0 | 2.0 | 2.0 | 5.0 | — | — | — | 0.5 | 0.5 |
| Sodium Cocoyl Hydrolyzed Wheat Protein | | | | | | | | | | |
| Euperlan ® PK 3000 AM | 5.0 | — | — | 5.0 | — | — | — | — | — | — |
| Glycol Distearate (and) Laureth-4 (and) | | | | | | | | | | |
| Cocamidopropyl Betaine | | | | | | | | | | |
| Arlypon ® F | — | — | — | — | — | — | — | — | — | — |
| Laureth-2 | | | | | | | | | | |
| Broccoli bud extract H1 | 1.0 | 1.0 | 1.0 | 1.0 | 1.0 | 1.0 | 1.0 | 1.0 | 1.0 | 1.0 |
| Hydagen ® CMF | 1.0 | 1.0 | 1.0 | 1.0 | 1.0 | 1.0 | 1.0 | 1.0 | 1.0 | 1.0 |
| Chitosan | | | | | | | | | | |
| Magnsium Sulfate Hepta Hydrate | — | — | — | — | — | — | — | — | 1.0 | 1.0 |
| Glycerin (85% by weight) | — | — | — | — | — | 3.0 | 3.0 | 5.0 | 5.0 | 3.0 |
| Composition (INCI) | 31 | 32 | 33 | 34 | 35 | 36 | 37 | 38 | 39 | 40 |
| Dehymuls ® PGPH | 4.0 | 3.0 | — | 5.0 | — | — | — | — | — | — |
| Polyglyceryl-2 Dipolyhydroxystearate | | | | | | | | | | |
| Lameform ® TGI | 2.0 | 1.0 | — | — | — | — | — | — | — | — |
| Polyglyceryl-3 Diisostearate | | | | | | | | | | |
| Emulgade ® PL 68/50 | — | — | — | — | 4.0 | — | — | — | 3.0 | — |
| Cetearyl Glucoside (and) Cetearyl Alcohol | | | | | | | | | | |
| Eumulgin ® B2 | — | — | — | — | — | — | — | 2.0 | — | — |
| Ceteareth-20 | | | | | | | | | | |
| Tegocare ® PS | — | — | 3.0 | — | — | — | 4.0 | — | — | — |
| Polyglyceryl-3 Methylglucose Distearate | | | | | | | | | | |
| Eumulgin VL 75 | — | — | — | — | — | 3.5 | — | — | 2.5 | — |
| Polyglyceryl-2 Dipolyhydroxystearate (and) | | | | | | | | | | |
| Lauryl Glucoside (and) Glycerin | | | | | | | | | | |
| Bees Wax | 3.0 | 2.0 | 5.0 | 2.0 | — | — | — | — | — | — |
| Cutina ® GMS | — | — | — | — | — | 2.0 | 4.0 | — | — | 4.0 |
| Glyceryl Stearate | | | | | | | | | | |
| Lanette ® O | — | — | 2.0 | — | 2.0 | 4.0 | 2.0 | 4.0 | 4.0 | 1.0 |
| Cetearyl Alcohol | | | | | | | | | | |
| Antaron ® V 216 | — | — | — | — | — | 3.0 | — | — | — | 2.0 |
| PVP/Hexadecene Copolymer | | | | | | | | | | |
| Myritol ® 818 | 5.0 | — | 10.0 | — | 8.0 | 6.0 | 6.0 | — | 5.0 | 5.0 |
| Cocoglycerides | | | | | | | | | | |
| Finsolv ® TN | — | 6.0 | — | 2.0 | — | — | 3.0 | — | — | 2.0 |
| C12/15 Alkyl Benzoate | | | | | | | | | | |
| Cetiol ® J 600 | 7.0 | 4.0 | 3.0 | 5.0 | 4.0 | 3.0 | 3.0 | — | 5.0 | 4.0 |
| Oleyl Erucate | | | | | | | | | | |
| Cetiol ® OE | 3.0 | — | 6.0 | 8.0 | 6.0 | 5.0 | 4.0 | 3.0 | 4.0 | 6.0 |
| Dicaprylyl Ether | | | | | | | | | | |
| Mineral Oil | — | 4.0 | — | 4.0 | — | 2.0 | — | 1.0 | — | — |
| Cetiol ® PGL | — | 7.0 | 3.0 | 7.0 | 4.0 | — | — | — | 1.0 | — |
| Hexadecanol (and) Hexyldecyl Laurate | | | | | | | | | | |
| Panthenol/Bisabolol | 1.2 | 1.2 | 1.2 | 1.2 | 1.2 | 1.2 | 1.2 | 1.2 | 1.2 | 1.2 |
| Broccoli extract H3 or H4 | 1.0 | 1.0 | 1.0 | 1.0 | 1.0 | 1.0 | 1.0 | 1.0 | 1.0 | 1.0 |
| Hydagen ® CMF | 1.0 | 1.0 | 1.0 | 1.0 | 1.0 | 1.0 | 1.0 | 1,0 | 1.0 | 1.0 |
| Chitosan | | | | | | | | | | |
| Copherol ® F 1300 | 0.5 | 1.0 | 1.0 | 2.0 | 1.0 | 1.0 | 1.0 | 2.0 | 0.5 | 2.0 |
| Tocopherol/Tocopheryl Acetate | | | | | | | | | | |
| Neo Heliopan ® Hydro | 3.0 | — | — | 3.0 | — | — | 2.0 | — | 2.0 | — |
| Sodium Phenylbenzimidazole Sulfonate | | | | | | | | | | |
| Neo Heliopan ® 303 | — | 5.0 | — | — | — | 4.0 | 5.0 | — | — | 10.0 |
| Octocrylene | | | | | | | | | | |
| Neo Heliopan ® BB | 1.5 | — | — | 2.0 | 1.5 | — | — | — | 2.0 | — |
| Benzophenone-3 | | | | | | | | | | |
| Neo Heliopan ® E 1000 | 5.0 | — | 4.0 | — | 2.0 | 2.0 | 4.0 | 10.0 | — | — |
| Isoamyl p-Methoxycinnamate | | | | | | | | | | |
| Neo Heliopan ® AV | 4.0 | — | 4.0 | 3.0 | 2.0 | 3.0 | 4.0 | — | 10.0 | 2.0 |
| Octyl Methoxycinnamate | | | | | | | | | | |
| Uvinul ® T 150 | 2.0 | 4.0 | 3.0 | 1.0 | 1.0 | 1.0 | 4.0 | 3.0 | 3.0 | 3.0 |
| Octyl Triazone | | | | | | | | | | |
| Zinc Oxide | — | 6.0 | 6.0 | — | 4.0 | — | — | — | — | 5.0 |
| Titanium Dioxide | — | — | — | — | — | — | — | 5.0 | — | — |
| Glycerol (86% by weight) | 5.0 | 5.0 | 5.0 | 5.0 | 5.0 | 5.0 | 5.0 | 5.0 | 5.0 | 5.0 |

(1-4) hair rinse, (5-6) conditioner, (7-8) shower bath, (9) shower gel, (10) wash lotion (11-14) “two-in-one” shower bath, (15-20) shampoo (21-25) foam bath, (26) soft cream, (27,28) moisturising emulsion, (29,30) night cream) (31) w/o sun protection cream, (32-34) w/o sun protection lotion, (35,38,40) o/w sun protection lotion, (36,27,39) o/w sun protection cream

1. Cosmetic and/or pharmaceutical preparations, characterized in that they contain
(a) an effective quantity of a Brassicaceae extract and
(b) oil components and/or emulsifiers and/or UV/IR protection factors and/or antioxidants.

2. Preparations as claimed in claim 1, characterized in that they contain broccoli, broccoli seed and/or broccoli bud extract.

3. Preparations as claimed in at least one of claims 1 and 2, characterized in that the extracts predominantly or exclusively contain glucoraphanin as active principle.

4. Preparations as claimed in at least one of claims 1 and 2, characterized in that the extracts predominantly or exclusively contain sulforaphane as active principle.

5. Preparations as claimed in at least one of claims 1 to 4, characterized in that the extracts predominantly or exclusively contain mixtures of glucoraphanin and sulforaphane as active principles.

6. Preparations as claimed in at least one of claims 1 to 5, characterized in that they contain the extracts in quantities of 0.1 to 10% by weight, based on the preparation.

7. Preparations as claimed in at least one of claims 1 to 6, characterized in that they contain
(a) 0.1 to 10% by weight of extracts and
(b) 1 to 99.9% by weight of oil components and/or 0.1 to 15% by weight of emulsifiers,
with the proviso that the the quantities shown optionally add up to 100% by weight with water and/or other auxiliaries and additives.

8. Preparations as claimed in at least one of claims 1 to 6, characterized in that they contain
(a) 0.1 to 10% by weight of a Brassicaceae extract and
(b) 0.1 to 20% by weight of UV/IR protection factors and/or antioxidants,
with the proviso that the quantities shown add up to 100% by weight with water and/or other auxiliaries and additives.

9. The use of Brassicaceae extracts as skin and hair care agents.

10. The use of Brassicaceae extracts as anti-inflammatory agents.

11. The use of Brassicaceae extracts as antimicrobial agents.

12. The use of Brassicaceae extracts as antioxidants.

13. The use of Brassicaceae extracts as agents for stimulating or regulating the formation of skin cells.

14. The use of Brassicaceae extracts as agents for stimulating skin detoxification enzymes.

15. The use of Brassicaceae extracts as anti-acne agents.

16. The use of Brassicaceae extracts as skin rejuvenating agents.

17. The use of Brassicaceae extracts as UV/IR protection factors.

18. The use of Brassicaceae extracts as agents against fibroblast and/or keratinocyte damage by UV radiation.

19. The use of Brassicaceae extracts as agents against UV-induced apoptosis and DNA damage.

20. The use claimed in any of claims 9 to 19, characterized in that broccoli, broccoli seed and/or broccoli bud extracts are used as the Brassicaceae extracts.

21. The use claimed in any of claims 9 to 19, characterized in that glucoraphanin and/or sulforaphane are used as the Brassicaceae extracts.
